(19) United States
(12) Patent Application Publication
(10) Pub. No.: US 2013/0317280 A1
(43) Pub. Date: Nov. 28, 2013

(54) MAGNETIC FIELD GENERATING SEAT CUSHION

(71) Applicant: Chen-Heng CHENG, Taichung City (TW)

(72) Inventor: Chen-Heng CHENG, Taichung City (TW)

(73) Assignee: BIOTEST MEDICAL CORPORATION, Taichung City (TW)

(21) Appl. No.: 13/477,206

(22) Filed: May 22, 2012

(51) Int. Cl. A61N 2/02

(52) U.S. Cl. 600/13

(57) ABSTRACT

A magnetic field generating seat cushion includes a cushion body, and a magnetic field generator, which is mounted in the cushion body, including a main circuit board, an inner coil and an outer coil. The inner and outer coils are electrically connected to the main circuit board. The main circuit board and the outer coil can generate an alternating current to go through the inner coil, causing the inner coil to generate an alternating magnetic field. In comparison to the outer coil, the inner coil has a high number of turns and a relatively smaller area for generating a high strength magnetic field in a small area with low electricity consumption to intermittently oxygenate the body tissues of a person sitting or lying on the cushion body, thereby generating a massage-like effect and achieving muscle-relaxing, blood-circulating and metabolism-enhancing effects.

### BACKGROUND OF THE INVENTION

1. Field of the Invention

The present invention relates to human health care products and more particularly, to a magnetic field generating seat cushion that can use electricity to generate a magnetic field for oxygenating the body tissues of a person sitting thereon, achieving healthcare effects.

2. Description of the Related Art

A seat cushion for providing health care effects by using a magnetic field is known comprising a cushion body and a plurality of magnetic stones. The magnetic stones are mounted inside the cushion body and can generate a continuous or static magnetic field to oxygenate the user's body tissues, achieving muscle-relaxing, blood-circulating and metabolism-enhancing effects.

However, it is difficult to adjust the direction or strength of a magnetic field generated by magnetic stones. Thus, the health care effect of a seat cushion of the aforesaid conventional design is limited.

### SUMMARY OF THE INVENTION

The present invention has been accomplished under the circumstances in view. It is the main object of the present invention that provides a magnetic field generating seat cushion, which can use electricity to generate a magnetic field for oxygenating the body tissues of a person sitting thereon, achieving muscle-relaxing, blood-circulating and metabolism-enhancing effects.

It is the other object of the present invention that provides a magnetic field generating seat cushion, which can intermittently generate a magnetic field for oxygenating the body tissues of a person sitting thereon, achieving better health care effects.

To achieve the aforesaid objects, a magnetic field generating seat cushion comprises a cushion body and a magnetic field generator mounted inside the cushion body. The magnetic field generator comprises a main circuit board, an inner coil electrically connected to the main circuit board, and an outer coil electrically connected to the main circuit board and surrounding the inner coil. The main circuit board is configured to generate a first alternating current together with the outer coil and capable of generating a second alternating current. The first alternating current and the second alternating current go alternatively through the inner coil to cause generation of an alternating magnetic field by the inner coil and the outer coil.

Further, the number of turns of the outer coil is smaller than the number of turns of the inner coil, and the frequency of the first alternating current is higher than the frequency of the second alternating current. The magnetic field generating seat cushion further comprises a controller disposed outside the cushion body and electrically connected with the main circuit board of the magnetic field generator. Further, the outer coil is capable of generating a magnetic field when electrically conducted.

Preferably, the magnetic field generator can intermittently generate an alternating magnetic field to provide better health care effects.

Other and further benefits, advantages and features of the present invention will be understood by reference to the following specification in conjunction with the accompanying drawings, in which like reference characters denote like elements of structure.

### BRIEF DESCRIPTION OF THE DRAWINGS

FIG. 1 is a perspective view of a magnetic field generating seat cushion in accordance with the present invention.

FIG. 2 is an elevational view of the magnetic field generator of the magnetic field generating seat cushion in accordance with the present invention.

### DETAILED DESCRIPTION OF THE INVENTION

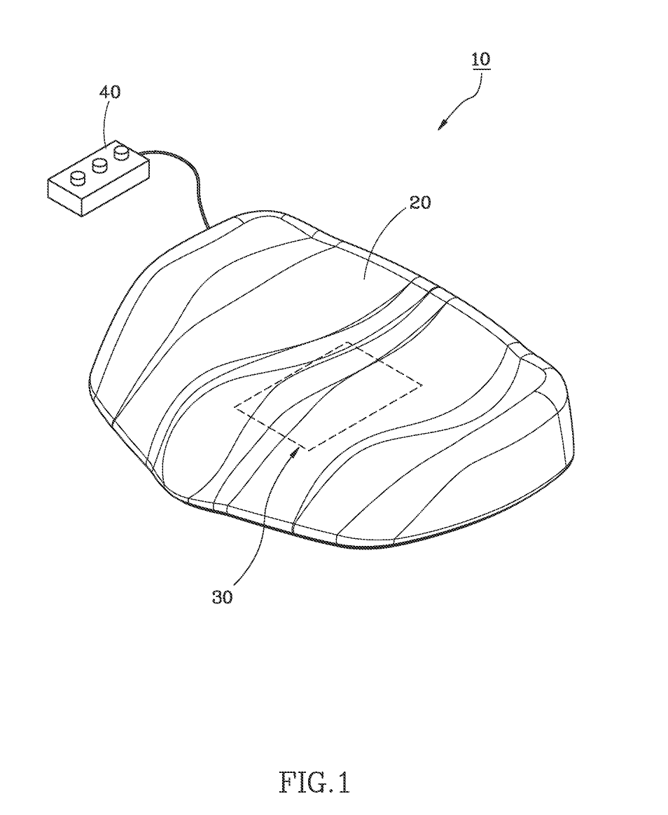

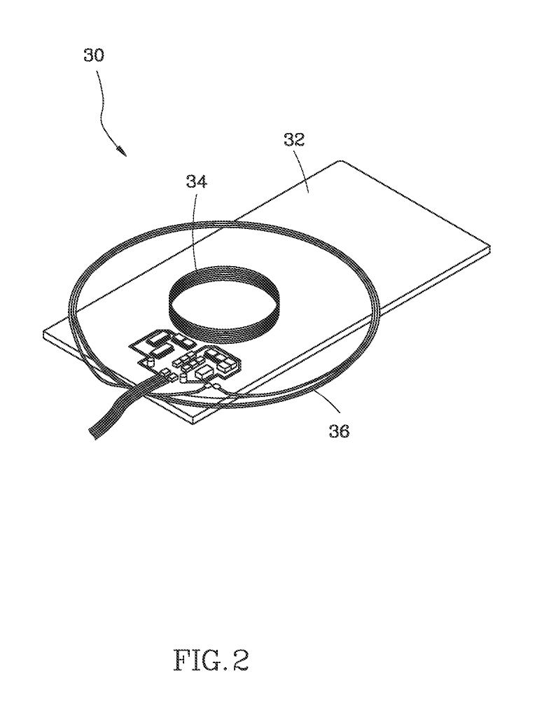

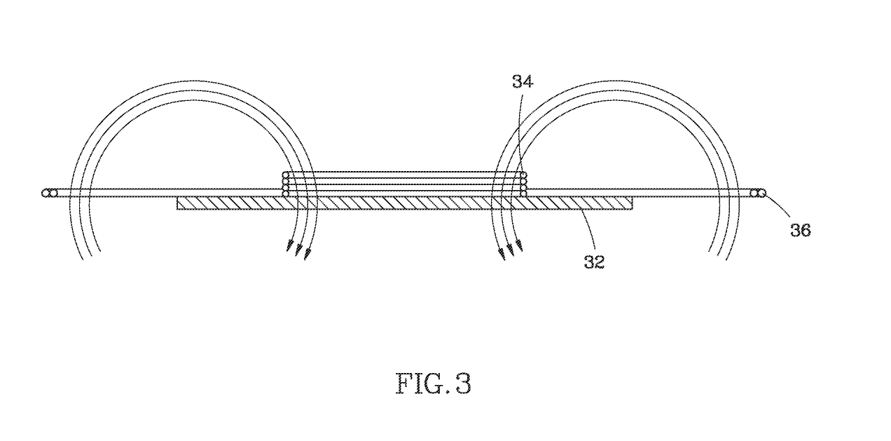

Referring to FIGS. 1 and 2, a magnetic field generating seat cushion 10 in accordance with the invention is shown. The seat cushion 10 comprises a cushion body 20, a magnetic field generator 30, and a controller 40.

The cushion body 20 is configured for a person to sit or lie thereon.

The magnetic field generator 30 is mounted inside the cushion body 20, comprising a main circuit board 32, an inner coil 34, and an outer coil 36. The main circuit board 32 is mounted inside the cushion body 20, carrying an IC, resistors, capacitors and a bipolar junction transistor (BJT). The inner coil 34 and the outer coil 36 are arranged inside the cushion body 20, and electrically connected to the main circuit board 32. The outer coil 36 surrounds the inner coil 34. Further, the number of turns of the outer coil 36 is smaller than the number of turns of the inner coil 34. The number of turns of the outer coil 36 can be 2 or 3. The number of turns of the inner coil 34 can be 100˜200.

When the magnetic field generator 30 is started, the main circuit board 32 can generate a first alternating current together with the outer coil 36, and the main circuit board 32 can generate a second alternating current independently. The first alternating current and the second alternating current go through the inner coil 34 alternatively, thereby causing the inner coil 34 to generate an alternating magnetic field showed as FIG. 3. The alternating magnetic field is semicircular and passes within the inner coil 34. The frequency of the first alternating current is higher than that of the second alternating current. In this embodiment, the first alternating current provides an oscillation frequency in the range of 17˜37 MHz and an amplitude in the range of 0.6˜1V; the second alternating current provides an oscillation cycle time in the range of 60˜140 mS and an amplitude in the range of 5˜10V.

It is to be noted that the main circuit board 32 sets the duration of the first alternating current or second alternating current in this embodiment to be 1 second, i.e., the first alternating current goes through the inner coil 34 for about one second, and then the second alternating current goes through the inner coil 34 after the first alternating current, and then the first alternating current goes through the inner coil 34 again. Thus, the first alternating current and the second alternating current are alternated through the inner coil 34 to generate an alternating magnetic field. This aforesaid duration of current can be adjusted by the designer subject to requirements. Preferably, the duration of current is in the range of 0.3 second to one minute. Further, the controller 40 is disposed outside the cushion body 20, and electrically connected with the main circuit board 32 of the magnetic field generator 30 for enabling a user to control the magnetic field generator 30.

Through the aforesaid structure, electricity can be conducted through the inner coil 34 and the outer coil 36 to generate a magnetic field for oxygenating the body tissues of a person sitting or lying on the cushion body 20, improving muscle relief, blood circulation and cellular metabolism relaxing. Further, because the inner coil 34 has a small dimension and a large number of turns, the magnetic field generator 30 can generate a high strength magnetic field in a small area with low electricity consumption to oxygenate the tissues of a particular part of the body (for example, prostate) of a person sitting or lying on the cushion body, enhancing the effects. Further, when the outer coil 36 is electrically conducted, the outer coil 36 can generate a low strength magnetic field in a large area to promote surrounding tissues.

It is to be noted that a time interval can be set during switching between the first alternating current and the second alternating current. During this time interval, the main circuit board generates no electric current, causing temporary interruption of the alternating magnetic fields. Thus, the magnetic field generator 30 can generate a periodically intermittent magnetic field to provide better health care effects.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A magnetic field generating seat cushion, comprising:
a cushion body; and
a magnetic field generator mounted inside said cushion body, said magnetic field generator comprising:
a main circuit board;
an inner coil electrically connected to said main circuit board; and
an outer coil electrically connected to said main circuit board and surrounding said inner coil;
wherein said main circuit board is configured to generate a first alternating current together with said outer coil and capable of generating a second alternating current, said first alternating current and said second alternating current going alternatively through said inner coil to cause generation of an alternating magnetic field by said inner coil and said outer coil.

2. The magnetic field generating seat cushion as claimed in claim 1, wherein the number of turns of said outer coil is smaller than the number of turns of said inner coil.

3. The magnetic field generating seat cushion as claimed in claim 1, wherein the frequency of said first alternating current is higher than the frequency of said second alternating current.

4. The magnetic field generating seat cushion as claimed in claim 1, further comprising a controller disposed outside said cushion body and electrically connected with said main circuit board of said magnetic field generator.

5. The magnetic field generating seat cushion as claimed in claim 1, wherein said outer coil is capable of generating a magnetic field when electrically conducted.

6. The magnetic field generating seat cushion as claimed in claim 1, wherein said main circuit board has set therein a time of duration for said first alternating current and said second alternating current.

7. The magnetic field generating seat cushion as claimed in claim 6, wherein said time of direction is in the range of 0.3 second to one minute.

8. The magnetic field generating seat cushion as claimed in claim 1, wherein said main circuit board has set therein a time interval for switching between said first alternating current and said second alternating current.
